(19) United States
(12) Patent Application Publication
(10) Pub. No.: US 2023/0126093 A1
(43) Pub. Date: Apr. 27, 2023

(54) SYNTHETIC DNA TEMPLATE FOR IN VITRO MRNA TRANSCRIPTION

(71) Applicants: Vrije Universiteit Brussel, Brussel (BE); myNEO NV, Gent (BE)

(72) Inventors: Kris Thielemans, Antwerpen (BE); Lorenzo Franceschini, Brussel (BE); Cedric Bogaert, Gent (BE)

(21) Appl. No.: 17/911,577

(22) PCT Filed: Mar. 16, 2021
(86) PCT No.: PCT/EP2021/056679

§ 371 Date: Sep. 14, 2022

(30) Foreign Application Priority Data
Mar. 16, 2020 (EP) 20163428.4

(51) Int. Cl. C12P 19/34; C12N 15/10

(52) U.S. Cl. CPC C12P 19/34; C12N 15/1068

(57) ABSTRACT

The present invention is related to the manufacturing of mRNA for use in immunotherapy or for use in immunotherapy screening. Compared to existing methods the present approach is plasmid-free, fast and inexpensive, allowing screening of potential neoantigen in immunotherapy.

### FIELD OF THE INVENTION

The present invention is related to the manufacturing of mRNA for use in immunotherapy or for use in immunotherapy screening or monitoring. Compared to existing methods the present approach is plasmid-free, fast and inexpensive, allowing screening of potential neoantigen in immunotherapy.

### BACKGROUND TO THE INVENTION

In for example cancer immunotherapy, the immune system is either passively or actively exploited to target and kill cancer cells. In this way, higher specificity for malignant cells may be achieved than with conventional cancer therapeutics. This approach thus avoids off-target toxicities while still inducing highly potent anti-cancer responses.

In active immunotherapy, immune cells are stimulated and instructed to actively fight cancer and although more challenging, this approach is extremely promising. Active immunotherapy is highly dependent on efficient stimulation of antigen-specific immune cells, such as killer T cells and antibody-producing B cells. One approach for in vivo induction of T-cell response is based on their interaction with antigen-presenting cells (APCs), in particular dendritic cells (DCs) that have been modified to have enhanced immunostimulatory characteristics when presenting the target-antigens at their surface.

In the modification of the antigen-presenting cells mRNA's encoding for immunostimulatory antigens are introduced in the APC requiring the manufacturing of the encoding mRNA's. In the present approach mRNA immunotherapy is based on the cloning of a target antigen peptide into a plasmid vector prior RNA production. This requires that the mRNA's encoding for the immunostimulatory antigens are combined as a string of antigens in a tandem minigene, cloned in a bacterial plasmid vector which is used for the in vitro transcription. This standard laboratory procedure requires long working protocols, including extensive plasmid screening and high cost reagents, causing the possible risk of bacterial contamination in the final product.

There is accordingly a need for an alternative method in the manufacturing of mRNA for use in immunotherapy or for use in immunotherapy screening or monitoring.

### SUMMARY OF THE INVENTION

In addressing the aforementioned problems, the present invention makes use of a plasmid free synthetic DNA template incorporating a single antigen instead, for the in vitro transcription and manufacturing of the mRNAs for use in immunotherapy or for use in immunotherapy screening.

The plasmid free synthetic DNA template (SDT) is prepared in an assembly PCR of 3 synthetic oligos, characterized in that said synthetic oligos comprise;
An oligo encoding an RNA polymerase (oligo 1);
An oligo encoding a 3′ untranslated region (3′ UTR) (oligo 3); and
An oligo encoding the antigen of interest containing bridging overlaps to the oligo encoding an RNA polymerase promotor and to the oligo encoding a 3′ UTR, respectively (oligo 2).

In one embodiment the RNA polymerase promotor used in oligo 1 is an RNA polymerase selected from the list comprising T7 promotor, SP6 promotor and T3 promotor; more in particular T7 promotor; even more in particular a modified RNA polymerase T7 promoter.

Oligo 3 comprises a 3′ untranslated region (3′ UTR) also known as trailer sequence which is important in translation termination as well as post-transcriptional modification of the transcript; in a particular embodiment oligo 3 comprises a 3′ UTR encoding a RNA stabilizer sequence such as the 3′ UTR from rabbit beta globin. For further stabilization of the mRNA, oligo 3 could further include a sequence encoding for a 3′ poly-A tail.

In one embodiment oligo 3 further comprises a trafficking domain that directs both membrane and non-membrane proteins to an endosomal compartment (e.g., a lysosome) in a cell; in particular a cytoplasmic endosomal/lysosomal targeting signal which effectively target antigens to that compartment. In a particular embodiment oligo 3 comprises the lumenal domain of a LAMP polypeptide, such as a LAMP-1, LAMP-2 polypeptide, or DC LAMP polypeptide.

In another embodiment oligo 1, further comprises a 5′ untranslated region (5′ UTR) and also a leader sequence or leader RNA for the regulation of translation of a transcript in eukaryotic cells; in particular the 5′ UTR encodes for a translation enhancer, such as the beta globulin enhancer promoter.

As proper trafficking of the encoded antigens in the antigen presenting cells is important in immunotherapy, further translation enhancers could be included in either of the synthetic oligos used in the manufacture of the synthetic DNA template. In a specific embodiment oligo 1 further comprises a Endoplasmic Reticulum (ER) signal sequence to promote the protein transfer into the ER, such as the signal sequence of human LMP1.

Thus in one aspect the present invention provides the plasmid free Synthetic DNA Template (SDT) obtained by assembly PCR of the 3 synthetic oligos as herein provided; more in particular the use of said plasmid free SDT in the manufacturing of mRNA for use in immunotherapy or for use in immunotherapy screening.

In another aspect the present invention provides a method of manufacturing mRNA for use in immunotherapy or for use in immunotherapy screening, said method comprising an assembly PCR of the 3 synthetic oligos as herein provided, yielding a plasmid free dsDNA assembly PCR product that corresponds to the plasmid free synthetic DNA template (SDT) for the in vitro transcription (iVT) of mRNA molecules. Hence, in one embodiment the method of manufacturing mRNA further comprises an iVT of the plasmid free SDT obtained from the assembly PCR reaction. Optionally the plasmid free dsDNA assembly PCR molecule is amplified by PCR prior to the iVT.

Thus in a particular embodiment the present invention provides a method of plasmid free manufacturing mRNA for use in immunotherapy or for use in immunotherapy screening, said method comprising;
comprising an assembly PCR of the 3 synthetic oligos as herein provided, yielding a plasmid free dsDNA assembly PCR product;
PCR amplification of said assembly PCR product, yielding a plasmid free SDT amplification product; and
Plasmid free iVT of said plasmid free SDT amplification product.

In one embodiment the plasmid free iVT of the SDT amplification product is performed using a nucleoside 5-triphosphate (NTP) mix with an RNA polymerase and polymerase A; in particular with an RNA polymerase selected from SP6, T3, and T7 RNA polymerase; more in particular with a nucleoside 5-triphosphate (NTP) mix with T7 RNA polymerase and polymerase A; for a time sufficient to yield the mRNA product. In the examples hereinafter the iVT was performed during 2 hours at 37° C.

In one embodiment the plasmid free iVT of the SDT amplification product, further comprises degradation of the SDT by Dnase treatment with LiCl precipitation of the mRNA product. Expressed differently, in the plasmid free iVT of the SDT amplification product, the mRNA product obtained from the reaction of the SDT with an NTP mix with an RNA polymerase and polymerase A; is further purified by a Dnase treatment (to degrade the SDT); in particular a Dnasl treatment, followed by a LiCl (2.5M) precipitation, with removal of the supernatant. The mRNA precipitate is washed with ice cold 70% ethanol, dried, dissolved in water, filtered, and stored at −80° C. before use.

### BRIEF DESCRIPTION OF THE DRAWINGS

FIG. 1: Top: A: CleanCap (cCAP) reagent AG for co-transcriptional capping of mRNA (m7G (5′) ppp (5′) (2′O MeA) pG), B: Structure of the SNA-mRNA. The mRNA encodes a single neoantigen flanked at its N-terminus by a signal peptide (SP) and at its C-terminus by an endosomal trafficking domain (DC-Lamp). The mRNA is stabilized by a 5′ and 3′ UTR and a poly(A) tail.

FIG. 2: Single neoantigen design. Each minigene encodes a 27-mer (27-mer in this figure represents a random sequence) which contains the mutated amino acid in a central position, flanked at its N terminal and COOH terminal side by 13 amino acids of the wild type sequence.

FIG. 3: Flow chart of the manufacturing process.

FIG. 4: Schematic diagram of the synthetic DNA template (SDT) (Intermediate I and II).

FIG. 5: SDT SNA-mRNA. Three synthetic deoxyribonucleic acid oligonucleotides (PAGE purified Ultramers, IDT) are assembled together via PCR assembling cycles (PAC) into a dsDNA molecule, which is further amplified by PCR for the generation of the plasmid free synthetic DNA template (SDT). The plasmid free SDT is used as such for in vitro transcription reaction (iVT) to generate plasmid free SNA-mRNA. Deoxyribonucleic acid oligonucleotide #1 (orange) & #3 (purple) are used in each SDT assembling PCR, containing standard sequences such as the T7 promoter, the 3′ & 5′ UTRs and the signal sequence derived from human Lamp1 and the endosomal trafficking domain from human DC-Lamp protein sequence respectively. oligonucleotide #2 (green) presents a 27mer neoantigen sequence, which is specific for each patient study.

FIG. 6 shows the diagram of the functional testing of single model antigens. The plasmid free single neoantigen mRNA (SNA-mRNA) is used to electroporate HLA-A2 expressing K562 cells. These cells were then co-cultured with CD8+T lymphocytes from an HLA-A2+ healthy donor that have been electroporated with the mRNA encoding the corresponding T cell receptor chains.

### DETAILED DESCRIPTION OF THE INVENTION

It is an object of the present invention to provide a method for plasmid free, i.e. without using plasmids, expression vectors and host organism, manufacturing mRNAs encoding antigen, herein also referred to as target specific antigens, typically used in in immunotherapy.

The term “target” used throughout the description is not limited to the specific examples that may be described herein. Any infectious agent such as a virus, a bacterium or a fungus may be targeted. In addition, any tumor or cancer cell may be targeted.

The term “target-specific antigen” used throughout the description is not limited to the specific examples that may be described herein. It will be clear to the skilled person that the invention is related to the induction of immunostimulation in antigen presenting cells, regardless of the target-specific antigen that is presented. The antigen that is to be presented will depend on the type of target to which one intends to elicit an immune response in a subject. Typical examples of target-specific antigens are expressed or secreted markers that are specific to tumor, bacterial and fungal cells or to specific viral proteins or viral structures.

The term “antigen presenting cell” used throughout the description includes all antigen presenting cells. Specific non limiting examples are dendritic cells, dendritic cell-lines, B-cells, or B-cell-lines. The dendritic cells or B-cells can be isolated or generated from the blood of a patient or healthy subject. The patient or subject can have been the subject of prior vaccination or not.

The terms “cancer” and/or “tumor” used throughout the description are not intended to be limited to the types of cancer or tumors that may have been exemplified. The term therefore encompasses all proliferative disorders such as neoplasma, dysplasia, premalignant or precancerous lesions, abnormal cell growths, benign tumors, malignant tumors, cancer or metastasis, wherein the cancer is selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma.

The plasmid free mRNA obtained using the method according to the invention, can be introduced in the antigen presenting cells, by electroporation, viral transduction (e.g. through lentivirus, adenovirus, or vaccinia virus), mRNA lipofection or DNA transfection. mRNA electroporation is especially preferred due to its high efficiency and its wide accepted use in clinical settings in contrast to viral transduction. For introduction of the target-specific antigens, pulsing of the cells with the antigen-specific peptides or with protein can be used as an alternative to mRNA electroporation. The introduced plasmid free mRNA can be a specifically synthesized sequence based on known tumor-specific markers, or can be isolated from (a) tumor cell line(s) or from a tumor-biopsy of the patient.

In the context of the present invention “mRNA” means “messenger RNA” and refers to a transcript which is produced using DNA as template and which itself codes for an antigen. An mRNA typically comprises a 5′-untranslated region, an antigen-encoding region and a 3′-untranslated region. mRNA has a limited half time in cells. According to the invention, mRNA will be prepared from a DNA template by in vitro transcription. It may be modified by further stabilizing modifications and capping, in addition to the modifications according to the invention. In the instant case the DNA-template is a synthetic DNA-template obtained through an assembly PCR of synthetic oligonucleotide sequences.

‘Synthetic oligonucleotides’ are chemically synthesized using building blocks called nucleoside phosphoramidites. These can be normal or modified nucleosides which have protecting groups to prevent their amines, hydroxyl groups and phosphate groups from interacting incorrectly. One phosphoramidite is added at a time, the 5′ hydroxyl group is deprotected and a new base is added and so on. The chain grows in the 3′ to 5′ direction, which is backwards relative to biosynthesis. At the end, all the protecting groups are removed. Nevertheless, being a chemical process, several incorrect interactions occur leading to some defective products. The longer the oligonucleotide sequence that is being synthesized, the more defects there are, thus this process is only practical for producing short sequences of nucleotides. The current practical limit is about 200 bp (base pairs) for an oligonucleotide with sufficient quality to be used directly for a biological application. HPLC can be used to isolate products with the proper sequence. Meanwhile, a large number of oligos can be synthesized in parallel on gene chips. For optimal performance in subsequent gene synthesis procedures they should be prepared individually and in larger scales.

Usually, a set of individually designed oligonucleotides is made on automated solid-phase synthesizers, purified and then connected by specific annealing and standard ligation or polymerase reactions. To improve specificity of oligonucleotide annealing, the synthesis step relies on a set of thermostable DNA ligase and polymerase enzymes. To date, several methods for gene synthesis have been described, such as the ligation of phosphorylated overlapping oligonucleotides, the Fok I method and a modified form of ligase chain reaction for gene synthesis. Additionally, several PCR assembly approaches have been described. They usually employ oligonucleotides of about 100 to 200 nucleotides long that overlap each other, typical overlaps of 10 to 30 bps are being used. These oligonucleotides are designed to cover most of the sequence of both strands, and the full-length molecule is generated progressively by overlap extension (OE) PCR, thermodynamically balanced inside-out (TBIO) PCR or combined approaches. The most commonly synthesized genes range in size from 600 to 1,200 bp although much longer genes have been made by connecting previously assembled fragments of under 1,000 bp. In this size range it is necessary to test several candidate clones confirming the sequence of the cloned synthetic gene by automated sequencing methods.

With regard to post-transcriptional regulation, it is has been demonstrated that certain 5′ and 3′ untranslated regions (UTRs) of eukaryotic mRNAs play a major role in translational efficiency and RNA stability, respectively. The 3′-untranslated region typically extends from the termination codon for a translation product, i.e. the antigen, to the poly-A sequence which is usually attached after the transcription process. The 3′-untranslated regions of mammalian mRNA typically have a homology region known as the AAUAAA hexanucleotide sequence. This sequence is presumably the poly-A attachment signal and is frequently located from 10 to 30 bases upstream of the poly-A attachment site. 3′-untranslated regions may contain one or more inverted repeats which can fold to give stem-loop structures, which act as barriers for exoribonucleases or interact with proteins known to increase RNA stability (e.g. RNA-binding proteins).

5′- and/or 3′-untranslated regions may, according to the invention, be functionally linked to a transcribable and in particular coding nucleic acid, such as signal peptide, promoter sequences, trafficking proteins and the like in such way that the stability and/or translation efficiency of the RNA that is transcribed from said transcribable antigenic nucleic acid are increased.

The poly-A tail as used in the context of this invention, preferably consists of between and about 100-150 adenosines, more in particular 120-125 adenosines, preferably >100 adenosines on average about 150 adenosines. The terms “poly-A tail” or “poly-A sequence” refer to a sequence of adenyl residues which is typically located at the 3′ end of an RNA molecule. The invention provides for such a sequence to be attached during RNA transcription by way of a DNA template on the basis of repeated thymidyl residues in the strand complementary to the coding strand, whereas said sequence is normally not encoded in the DNA but is attached to the free 3′ end of the RNA by a template-independent RNA polymerase after transcription in the nucleus. According to the invention, a poly(A) sequence of this kind is understood as meaning a nucleotide sequence of at least 20, preferably at least 40, preferably at least 80, preferably at least 100 and preferably up to 500, preferably up to 400, preferably up to 300, preferably up to 200, and in particular up to 150 consecutive A nucleotides, and in particular about 120 consecutive A nucleotides, wherein the term “A nucleotides” refers to adenyl residues.

### Examples

Description of Synthetic mRNA Encoding a Single Tumor Specific Antigen

Plasmid free synthetic mRNA encoding one tumor-specific neoantigen has been prepared. The synthetic mRNA encodes for a signal peptide, a neoantigen peptide, linked to an endosomal targeting protein designed for optimal processing and presentation of the encoded epitopes in the context of HLA class-I and -II. Each antigen sequence encodes for an oligopeptide of about 27 amino acids. Evidently the size of the antigenic oligopeptide can vary and is typically in the range of about 20 to about 50 amino acids. The mRNA is stabilized by a 5′ and 3′ untranslated regions (UTRs) and an extensive polyadenylated region named poly(A) tail.

In what follows these plasmid free synthetic mRNA's encoding one tumor-specific neoantigen are also being referred to as;
Descriptive name: Single neoantigen mRNA (SNA-mRNA)
Short description: Synthetic mRNA encoding one neoantigen
Firm or lab code: SNA-mRNA

Structure of the SNA-mRNA's

Messenger ribonucleic acid (mRNA) consists of a single-stranded polymer of 4 nucleotides: adenosine, guanosine, cytidine and uridine monophosphate with a 5′-end Cap structure and a 3′-end poly-A tail. The nucleotides are linked to each other through the 3′ and 5′ phosphate residues of the ribose sugars

The structure of the plasmid free in vitro synthesized mRNA manufactured by LMCT consists of the following elements:
5′ cap1 structure
5′ translation enhancer (TE) (5′UTR)
Open reading frame (ORF)
3′ RNA stabilizer sequence (3′ UTR)
a 3′ poly-A tail.

In the cytoplasm, the ORF corresponds to the coding region of the Gene of Interest (GoI) and will be translated into a polymer of amino acids, a protein.

The Cap1 modification forces the RNA polymerase to start transcription at the 3′ OH group of the unmethylated guanosine (G). The ORF encodes a fusion protein consisting of a signal peptide (SP), a 27 amino acid long polypeptide corresponding to a neoantigen and an endosomal trafficking domain derived from dendritic cell-lysosomal associated membrane protein (DC-LAMP/CD208) (FIG. 1). The ORF is flanked by a 5′ and 3′ untranslated regions (UTR) and followed by a poly(A) tail of 120 adenosine residues (A120) or 150 adenosine residues (A150). The UTRs and poly(A) tail of 120 or 150 nucleotides long contribute both to the stability and translational efficiency of the mRNA.

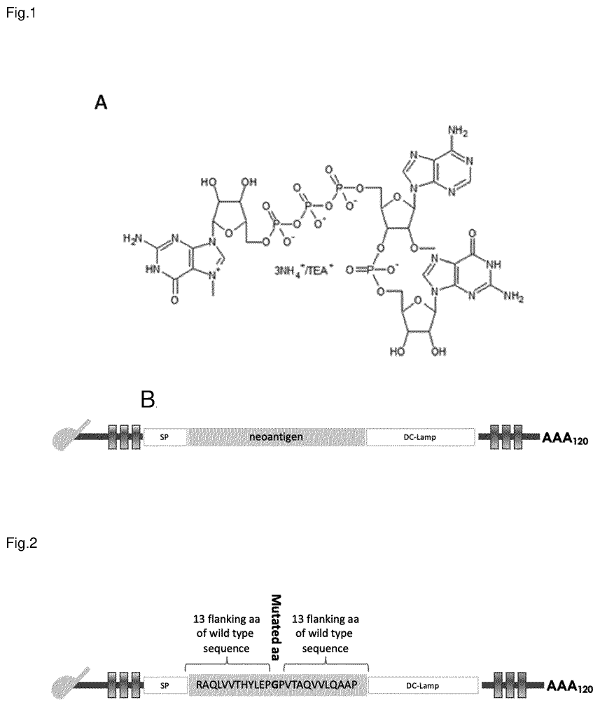

The neoantigen sequence is different for each study subject (FIG. 2). In case of a single nucleotide variant (SNV), the minigene encodes a 27-mer consisting of the mutated amino acid flanked at both sides by 13 amino acids of the wild type protein. In case of an indel-frame shift mutation, the minigene encodes a 27-mer that includes the frameshifted sequence predicted to bind to an HLA allotype of the patient.

General Properties

Messenger RNA is highly sensitive to RNases and should always be treated with extreme care using only materials of the highest purity (endotoxins and nucleases should be avoided) during manufacturing and manipulations. Skin surface and serum are both high sources of RNases and contact with the mRNA should be avoided at all times.

Plasmid free messenger RNA is very stable in aqueous solutions when stored below −15° C.

A major advantage of the use of plasmid free mRNA is its safety profile. RNA has a very short half-life time in vivo leading to only transient translation into protein until ubiquitous RNases will degrade the mRNA molecules. Moreover, mRNA does not need to cross the nuclear envelope to be translated into protein and cannot be integrated into the host genome of the cell that captured the mRNA, as opposed to plasmid DNA or DNA containing viral vectors.

SNA-mRNA is mRNA and as such is thought to be rapidly degraded in vivo similarly to natural mRNA.

The function of the various components of the SNA-mRNA are as follows (Bonehill 2004): The SP and DC.Lamp encoded amino acids route the fusion protein to the endoplasmic reticulum and the endolysosomal pathway, thereby enabling degradation of the protein and loading into HLA class II molecules. In addition, due to endoplasmatic reticulum (ER) stress response, the protein will be partially rerouted to the cytoplasm and the proteasome thereby ensuring also efficient HLA class I presentation. The 5′ and 3′UTRs are designed for optimal stability. A poly-A tail of 120 nucleotides ensures optimal stability. SNA-mRNA is electroporated in DC during the production process of the drug substance. Shortly after electroporation (within minutes), the mRNA is translated into protein, the protein inserts itself in the ER and is subsequently processed in the class I (proteasome) and the class II (endolysosomal) antigen processing pathways. The design of the 27-mer epitopes is such that, based on present knowledge, it is predicted that 8-mer, 9-mer or 10-mer peptides will be generated that bind the HLA-A, -B, -C (class I) alleles and/or 12-16-mer peptides that bind to the HLA class II alleles of the patient. Using reporter constructs with a similar design (FIG. 1), we could show that within 4 h the intact protein (Green fluorescent protein (GFP) reporter) is expressed in the cells and subsequently degraded in a lysosomal compartment (Bonehill 2004).

Similarly, peptides are processed and presented in the context of HLA membrane proteins (using e.g. p53 and gp100 epitopes encoded by a mRNA of similar design as shown in FIG. 6).

Neoepitope peptides bound to HLA on the patient DCs are “foreign” to the patient (i.e. are not present in none of the tissues of the patient before the tumor arose) and are expected to activate the CD4 and/or CD8 T cells of the patient and differentiate them to T helper 1 (TH1) cells producing interferon gamma (IFN-□) and to cytotoxic killer T cells (CTL), respectively. In the days and weeks following vaccination, these T cells are expected to migrate to the tumor tissue and selectively kill the tumor cells by various immune mechanisms. The T cells may survive as memory cells and continue to protect the patient against tumor relapse.

Description of Manufacturing Process and Process Controls

The manufacturing process of the starting material can be divided in two parts:
Part I: Production of plasmid free synthetic DNA template (SDT) (=intermediate 1)
   Assembly PCR of 3 synthetic ssDNA oligonucleotides and initial formation of the dsDNA template, purification and sequencing
   Plasmid free amplification of the sequence verified IM1 (=intermediate 2)
Part II: Plasmid free production of mRNA and purification
   plasmid free in vitro transcription of SDT into mRNA, purification and vialing

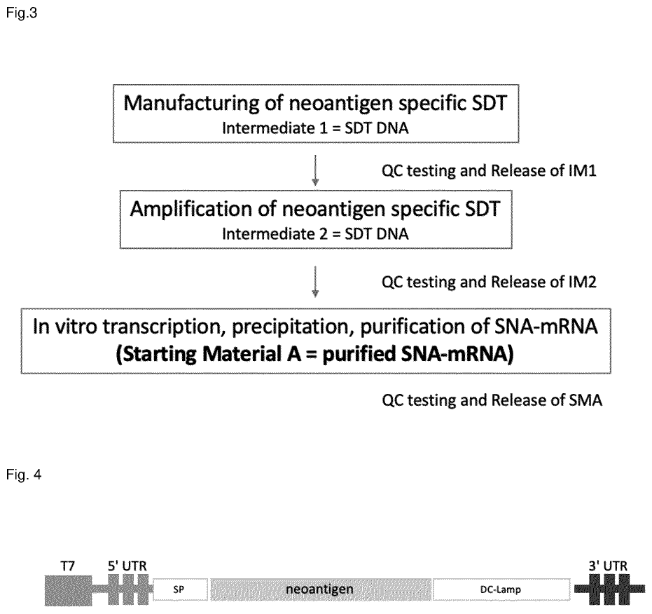

A flow chart of the manufacturing process of starting material A is illustrated in FIG. 3.

The starting material is a synthetic DNA template (SDT, FIG. 4), containing the DNA sequence of modified T7 RNA polymerase promotor, followed by a beta-globin translation enhancer sequence as 5′ UTR. The open reading frame (ORF) includes the single neoantigen sequence of interest placed between the Lamp1 signal peptide (SP) and the endosomal trafficking domain DC-LAMP sequences followed by downstream a beta-globin 3′ UTR region.

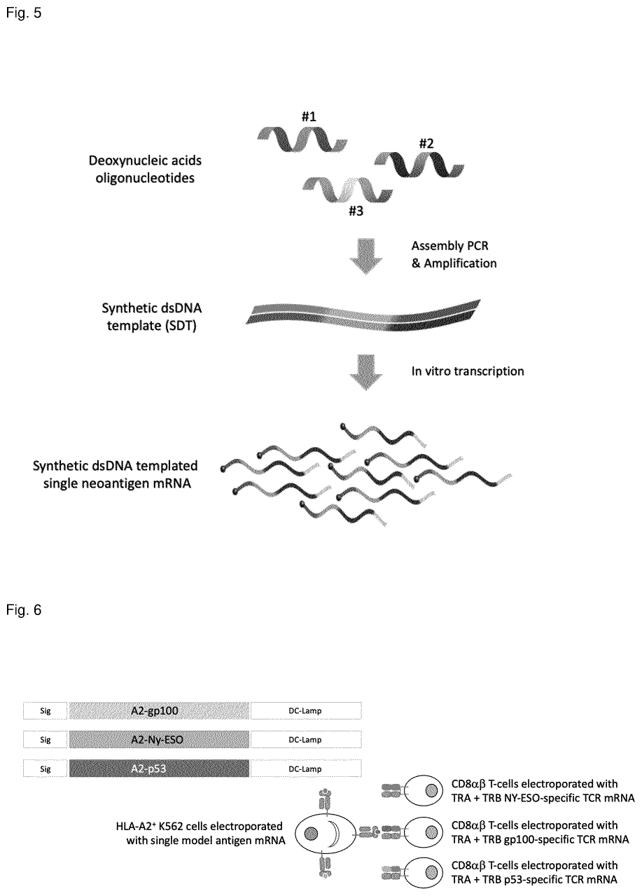

FIG. 5 shows the stepwise production of SDT SNA-mRNA. Three synthetic deoxyribonucleic acid oligonucleotides (Ultramers, IDT) are designed to hybridize together in an assembly polymerase reaction (aPCR), forming a dsDNA template for the plasmid free in vitro transcription (iVT) of mRNA molecules. The 3 ssDNA oligonucleotides show all the fundamental features for high cellular stability and translatability of the transcribed mRNA, and moreover enhanced antigen membrane presentation of the transcribed single neoantigen peptide. In particular, oligonucleotide #1 encodes for the modified RNA polymerase T7 promoter, a beta globin enhancer promoter for efficient protein translation as 5′ untranslated region (UTR) and the signal sequence from human Lamp1. Oligonucleotide #2 can encode for any neoantigen sequence required and contains sequences overlapping with oligonucleotide #1 and #3. Oligonucleotide #3, introduces an endosomal trafficking domain from DC-LAMP for class-I & -II antigen presentation and the 3′ untranslated region (UTR) from rabbit beta globin. Oligonucleotide #1 and #3 provide the basic mRNA sequence features and are used for each SDT SNA-mRNA production, while Oligonucleotide #2 can be de novo synthetized for each patient-specific neoantigen sequence to be tested.

Main steps in the production of SDT SNA-mRNA are:
1. Assembly PCR and SDT formation, purification and identification via sequencing analysis and capillary gel electrophoresis (intermediate I).
2. A sequence verified SDT batch is further amplified according to the quantity needed for the mRNA production (intermediate II).

After purification, sterile filtration and extensive quality controls, the plasmid free SDT is used for in vitro mRNA synthesis.

Main steps in the aseptic production of mRNA:
1. In vitro transcription of mRNA and subsequent removal of residual SDT DNA
2. Purification of the mRNA followed by filtration and aseptic vialing and storage of the SNA mRNA

In vitro transcription, precipitation and purification of SDT SNA-mRNA: SDT SNA-mRNA is produced in batches starting from synthetic DNA templates (SDT). After addition of CleanCap/NTP mix and T7 RNA polymerase, in vitro transcription is performed during 2 hours at 37° C. The reaction mix also contains polymerase A for the poly-A tailing of the mRNA. Subsequently, SDT is degraded by Dnasel treatment and the mRNA is purified using LiCl precipitation. After two wash steps, pelleted mRNA is dissolved in water for injection. The batch is filtered and aseptically filled in cryovials and subsequently stored at −80° C. awaiting results of the QC testing.

In Vitro Assay

To test the functionality of the resulting mRNAs we performed ‘antigen presentation assays.

To this end, the Single Model Antigen mRNAs were electroporated into HLA-A2 expressing K562 cells. These cells were then co-cultured with CD8+T lymphocytes from an HLA-A2+ healthy donor that had been electroporated with the mRNA encoding the corresponding T cell receptor chains (as schematically shown in FIG. 6). Upon recognition of their corresponding antigenic epitope, the cells will secrete IFN-γ.

| | CD8αβ T cells electroprated with | | | | | |
|---|---|---|---|---|---|---|
| K562-A2 cells | gp100 TCR | | NY-ESO-TCR | | p53 TCR | |
| electroporated with: | mRNA | | mRNA | | mRNA | |
| Experiment 1 | | | | | | |
| Ctr mRNA | 57.05 | pg | 44.87 | pg | 78.81 | pg |
| Mix of single model | 2.182.27 | pg | 1.480.31 | pg | 294.51 | pg |
| antigen mRNA | | | | | | |
| (3x 0.5 μg) | | | | | | |
| TMG-mRNA (1 μg) | 354.53 | pg | 234.50 | pg | 651.505 | pg |
| Experiment 2 | | | | | | |
| Ctr mRNA | 8.72 | pg | 6.67 | pg | 15.02 | pg |
| Mix of single model | 76.02 | pg | 136.25 | pg | 45.09 | pg |
| antigen mRNA | | | | | | |
| (3x 0.5 μg) | | | | | | |
| TMG-mRNA (1 μg) | 62.36 | pb | 107.18 | pg | 130, 147 | pg |
| Experiment 3 | | | | | | |
| Ctr mRNA | 0.41 | pg | 3.97 | pg | 7.70 | pg |
| Mix of single model | 103.24 | pg | 77.78 | pg | 135.07 | pg |
| antigen mRNA | | | | | | |
| (3x 0.5 μg) | | | | | | |
| TMG-mRNA (1 μg) | 280.85 | pg | 59.45 | pg | 173.66 | pg |

In the aforementioned table the results for a mixture of corresponding SNA-mRNA's are comparable with a tandem minigene (e.g. 5 neoantigens of 27 amino acids each encoded by 81 nucleotides), the open reading frame of which will be 405 bp long and has to be cloned into a bacterial plasmid (pLMCT) vector. pLMCT-TMG DNA is used as template for the in vitro transcription.

1. A plasmid free synthetic DNA template (SDT) prepared in an assembly PCR of 3 synthetic oligos, characterized in that said synthetic oligos comprise;
An oligo encoding an RNA polymerase promotor (oligo 1);
An oligo encoding a 3′ untranslated region (3′ UTR) (oligo 3); and
An oligo encoding the antigen of interest containing bridging overlaps to the oligo encoding an RNA polymerase and to the oligo encoding a sequence for antigen presentation, respectively (oligo 2).

2. The SDT according to claim 1, wherein the RNA polymerase promotor used in oligo 1 is an RNA polymerase promotor selected from the list comprising T7 promotor, SP6 promotor and T3 promotor; more in particular T7 promotor; even more in particular a modified RNA polymerase T7 promoter.

3. The SDT according to claim 1, wherein oligo 3 comprises a 3′ UTR encoding a RNA stabilizer sequence such as the 3′ UTR from rabbit beta globin.

4. The SDT according to claim 1, wherein oligo 3 could further include a sequence encoding for a 3′ poly-A tail.

5. The SDT according to claim 2, wherein oligo 1 further comprises a 5′ untranslated region (5′ UTR) also known as a leader sequence or leader RNA for the regulation of translation of a transcript in eukaryotic cells; in particular the 5′ UTR encodes for a translation enhancer, such as the beta globulin enhancer promoter.

6. The SDT according to claim 3, wherein oligo 3 further comprises a sequence for antigen presentation such as a trafficking domain that directs both membrane and non-membrane proteins to an endosomal compartment (e.g., a lysosome) in a cell; in particular a cytoplasmic endosomal/lysosomal targeting signal which effectively target antigens to that compartment; In a particular embodiment oligo 3 comprises the lumenal domain of a LAMP polypeptide, such as a LAMP-1, LAMP-2 polypeptide, or DC LAMP polypeptide.

7. The SDT according to claim 2, wherein oligo 1 further comprises a Endoplasmic Reticulum (ER) signal sequence to promote the protein transfer into the ER, such as the signal sequence of human LMP1.

8. Use of SDT according to any one of claims 1 to 7 in plasmid free manufacturing of mRNA for use in immunotherapy or for use in immunotherapy screening.

9. An in vitro method of plasmid free manufacturing mRNA for use in immunotherapy or for use in immunotherapy screening, said method comprising an assembly PCR of the 3 synthetic oligos as defined in the preceding claims, yielding a plasmid free dsDNA assembly PCR product.

10. The method according to claim 9, further comprising a plasmid free in vitro transcription (iVT) of the dsDNA assembly PCR product.

11. The method according to claim 10, wherein the dsDNA assembly PCR molecule is amplified by PCR prior to the iVT.

12. The method according to claim 10, wherein the plasmid free in vitro transcription (iVT) of the dsDNA assembly PCR product comprises contacting said dsDNA assembly PCR product with a nucleoside 5-triphosphate (NTP) mix with an RNA polymerase and polymerase A; in particular with an RNA polymerase selected from SP6, T3, and T7 RNA polymerase; more in particular with a nucleoside 5-triphosphate (NTP) mix with T7 RNA polymerase and polymerase A; for a time sufficient to yield the mRNA product.

13. The method according to claim 12, further comprising purifying the mRNA product by a Dnase treatment (to degrade the SDT); in particular a Dnasl treatment, followed by a LiCl (2.5M) precipitation, with removal of the supernatant.

14. An in vitro method of plasmid free manufacturing mRNA for use in immunotherapy or for use in immunotherapy screening, said method comprising;
comprising an assembly PCR of the 3 synthetic oligos as defined in the preceding claims, yielding a dsDNA assembly PCR product;
PCR amplification of said assembly PCR product, yielding a plasmid free SDT amplification product; and
Plasmid free iVT of said SDT amplification product; in particular in according with the iVT as described in claims 12 and 13.
